(19) United States
(12) Patent Application Publication
(10) Pub. No.: US 2008/0058429 A1
(43) Pub. Date: Mar. 6, 2008

(54) Diagnosis Of Primary Open Angle Glaucoma

(76) Inventors: A Louis Southren, Monsey, NY (US); Bernard I. Weinstein, New York, NY (US); Victor A. Fried, Katonah, NY (US)

Correspondence Address: CLARK & ELBING LLP, 101 FEDERAL STREET, BOSTON, MA, 02110, US

(21) Appl. No.: 10/545,455

(22) PCT Filed: Feb. 13, 2004
(86) PCT No.: PCT/US04/04429

§ 371 Date: Apr. 28, 2006

Related U.S. Application Data
(60) Provisional application No. 60/447,281, filed on Feb. 14, 2003.

(51) Int. Cl. A61K 47/00; A61P 43/00; G01N 33/53

(52) U.S. Cl. 514/789; 435/7.1

(57) ABSTRACT

The invention provides methods for diagnosing patients with primary open angle glaucoma (POAG), as well as methods for identifying agents for use in treating POAG and methods of using such agents in prevention and treatment of the disease.

### BACKGROUND OF THE INVENTION

Glaucoma is a chronic disease that is typically characterized by increased intraocular pressure and associated damage to the optic nerve. If untreated, glaucoma results in progressive loss of peripheral vision, followed by loss of central vision and, ultimately, blindness. Primary Open Angle Glaucoma (POAG) is the most common form of glaucoma in the Western world and affects about 2% of the population over the age of 40 years. Effective treatment for POAG is available, but is not always sought, particularly because the early stages of the disease are not usually associated with any symptoms. Indeed, when symptoms do become apparent, irreversible damage is likely to have already occurred, and thus it may be too late to provide effective treatment. Early diagnosis of POAG is therefore essential to ensure the availability of optimal treatment.

Approaches for diagnosing POAG that are generally in use today involve assessment of a patient's visual field, intraocular pressure, and appearance of the optic disc, taken in consideration with risk factors such as family history, age, and race. These approaches require a highly skilled medical professional for proper evaluation, and are time consuming and expensive. We previously developed a method for diagnosing POAG based on our discovery that decreased activity of 3α-hydroxysteroid dehydrogenase (3α-HSD) enzyme in trabecular meshwork cells of the eyes of POAG patients, as compared to unaffected individuals, correlates with a detectable decrease in 3α-HSD enzyme activity in peripheral blood lymphocytes (U.S. Pat. No. 5,376,534). Methods such as this, involving analysis of a blood sample from a patient, are relatively simple, inexpensive, and rapid, and therefore are highly desirable.

### SUMMARY OF THE INVENTION

The present invention provides new methods for determining whether a subject has or is at risk of developing primary open angle glaucoma (POAG), as well as methods for identifying and using agents effective in the prevention and treatment of POAG. The diagnostic methods involve contacting a sample (e.g., whole blood, plasma, serum, blood cells (e.g., peripheral blood lymphocytes), urine, saliva, or epithelial cells) from a subject with an antibody specific for 3α-hydroxysteroid dehydrogenase (3α-HSD), and classifying the subject as having or being at risk for developing POAG or not, based on the level of immunoreactivity of the antibody with 3α-HSD present in the sample. Optionally, a control can be processed in parallel with the sample from the subject, as is described elsewhere herein.

In one example of the methods of the invention, the antibody used for diagnosis is specific for the form of 3α-HSD present in subjects who have or are at risk of developing POAG, and detection of increased immunoreactivity in the sample with the antibody, relative to a control based on the form of 3α-HSD present in unaffected subjects, indicates a diagnosis of POAG or a risk thereof.

In another example, the antibody is specific for the form of 3α-HSD present in subjects who have or are at risk of developing POAG, and detection of decreased immunoreactivity in the sample with the antibody, relative to a control based on the form of 3α-HSD present in subjects who have or are at risk of developing POAG, indicates that the subject does not have and is not at risk of developing POAG.

In a further example, the antibody is specific for the form of 3α-HSD present in subjects who do not have or are not at risk of developing POAG, and detection of decreased immunoreactivity in the sample with the antibody, relative to a control based on the form of 3α-HSD present in unaffected subjects, indicates a diagnosis of POAG or a risk thereof.

In yet another example, the antibody is specific for the form of 3α-HSD present in subjects who do not have or are not at risk of developing POAG, and detection of increased immunoreactivity in the sample with the antibody, relative to a control sample based on the form of 3α-HSD present in subjects who have or are at risk of developing POAG, indicates that the subject does not have and is not at risk of developing POAG.

Any of a number of different formats can be used to carry out the methods of the invention. For example, the methods can involve a format in which the sample is fractionated by gel electrophoresis and transferred to a membrane prior to contact with the antibody. In another example, the antibody is bound to an adherent medium (e.g., a well of an assay plate or a plastic strip), the sample is contacted with the antibody on the adherent medium, and binding of 3α-HSD of the sample to the antibody on the adherent medium is detected by use of a detectably labeled antibody that specifically binds to 3α-HSD. In a further example, any complexes formed between the antibody and 3α-HSD in the sample are detected after chromatographic separation of the complexes from uncomplexed antibody. Additional details of these assay formats are provided below.

The invention also provides kits for use in determining whether a subject has or is at risk of developing POAG. The kits of the invention include one or more antibodies specific for 3α-HSD, for example, an antibody specific for the form of 3α-HSD present in subjects who have or are at risk of developing POAG, and/or an antibody specific for the form of 3α-HSD present in subjects who do not have or are not at risk of developing POAG. Optionally, the kits can also include one or more standards, for example, a standard including 3α-HSD in the form that is present in subjects who do not have or are not at risk of developing POAG, and/or a standard including 3α-HSD in the form that is present in subjects who have or are at risk of developing POAG. Further, the list can include an adherent medium (e.g., an assay plate or a plastic strip) to which the antibody binds, or a chromatographic medium for distinguishing complexes formed between 3α-HSD and the antibody and free antibody. The kits of the invention can also include one or more detectably labeled secondary antibodies, as is described further below.

The invention further provides methods of identifying agents that can be used in the prevention or treatment of POAG. In these methods, a trabecular meshwork cell (e.g., a cultured cell or a cell in an animal) from a subject with POAG is contacted with a candidate agent, which is then classified as being effective in the treatment or prevention of POAG, based on the effect of the agent on the level of immunoreactivity of an antibody specific for 3α-HSD with 3α-HSD of the cell. In one example of these methods, the antibody specific for 3α-HSD is specific for the form of 3α-HSD present in subjects who have or are at risk of developing POAG, and detection of a decreased level of immunoreactivity of the antibody with 3α-HSD of the cell, in the presence of the candidate agent, indicates the identification of an agent that can be used in the prevention or treatment of POAG. In another example, the antibody specific for 3α-HSD is specific for the form of 3α-HSD present in subjects who do not have or are not at risk of developing POAG, and detection of an increased level of immunoreactivity of the antibody with 3α-HSD of the cell, in the presence of the candidate agent, indicates the identification of an agent that can be used in the prevention or treatment of POAG.

Also included in the invention are methods of identifying agents that can be used in the prevention or treatment of POAG, which involve administering a candidate agent to a subject having POAG or a model condition thereof, and classifying the agent as being effective in the treatment or prevention of POAG, based on the effect of the agent on the level of immunoreactivity of an antibody specific for 3α-HSD with a sample from the subject. In one example of these methods, the antibody specific for 3α-HSD is specific for the form of 3α-HSD present in subjects who have or are at risk of developing POAG, and detection of a decreased level of immunoreactivity of the antibody with 3α-HSD in the sample, in the presence of the candidate agent, indicates the identification of an agent that can be used in the prevention or treatment of POAG. In another example, the antibody specific for 3α-HSD is specific for the form of 3α-HSD present in subjects who do not have or are not at risk of developing POAG, and detection of an increased level of immunoreactivity of the antibody with 3α-HSD in the sample, in the presence of the candidate agent, indicates the identification of an agent that can be used in the prevention or treatment of POAG. Further, in these methods, the sample tested can be selected from the group consisting of whole blood, plasma, serum, blood cells (e.g., peripheral blood lymphocytes), urine, saliva, and epithelial cells.

The invention also includes methods of preventing or treating POAG in a subject, involving administration (e.g., ocular administration) of an agent that is identifiable as being effective in such prevention and treatment using the screening methods described herein.

The terms “increased” or “decreased” as used herein are used in reference to a control. As is discussed further below, changes in 3α-HSD levels can be determined by comparison to a control that is processed in parallel to a patient sample. Alternatively, such parallel processing of a control is not necessary if a diagnostic level of 3α-HSD has previously been determined, and the assay has been adjusted to be sensitive to this level. Diagnostic changes in the level of detection of 3α-HSD in a sample, relative to a control, can be, for example, as low as 1.5 to 2-fold, or as much as 5-fold, 10-fold, 25-fold, 50-fold, or 100-fold or more, depending on the assay format used.

By “3α-HSD equivalent to that from a subject who does not have or is not at risk of developing POAG” is meant 3α-HSD obtained from such a subject or 3α-HSD that is not immunologically distinct from such material, using an assay method such as one of those described herein. Thus, in addition to being obtained from an unaffected subject, such 3α-HSD can be from another tissue source or species, or can be recombinantly made, provided that antibodies raised to it are capable of detecting a difference in the enzyme between POAG and non-POAG samples. As a specific example, which is described further below, we have found that an antibody raised to 3α-HSD purified from rat liver can be used to distinguish 3α-HSD from POAG vs. non-POAG patient samples. Similarly, by “3α-HSD equivalent to that from a subject who has or is at risk of developing POAG” is meant 3α-HSD that is obtained from such a subject or is not immunologically distinct from such material, using assay methods such as those described herein.

The invention provides several advantages. For example, the methods of the invention do not require the time consuming, expensive analysis of a highly skilled medical professional, which is required in the current, widespread approaches to diagnosis. Further, the assay formats made possible by the present invention are immunologically based, and such assays are by their nature simple, rapid, and inexpensive, in comparison to assays requiring the analysis of enzymatic activity, such as the assay noted above (U.S. Pat. No. 5,376,534, the teachings of which are incorporated herein by reference). In addition, the methods of the present invention can be carried out using frozen samples, in contrast to enzyme assay-based methods. The invention thus provides rapid, efficient, and cost effective approaches to diagnosing POAG. These improvements are highly significant, as they will help to facilitate early diagnosis, which, as is discussed above, is essential for enabling optimal treatment.

Other features and advantages of the invention will be apparent from the following detailed description, the drawings, and the claims.

### BRIEF DESCRIPTION OF THE DRAWINGS

FIG. 1A is an immunoblot of samples from non-POAG (lanes 1-3) and POAG (lanes 4-6) patients stained with an antibody to 3α-HSD.

FIG. 1B is a Coomassie-stained gel of samples from non-POAG (lanes 1-3) and POAG (lanes 4-6) patients.

### DETAILED DESCRIPTION

The invention provides methods and kits for use in determining whether a subject has or is at risk of developing primary open angle glaucoma (POAG). Also provided by the invention are methods of identifying agents that can be used in the prevention and treatment of POAG, as well as methods of preventing and treating POAG using such agents. The invention is based on the present inventors' discovery that the enzyme 3α-hydroxysteroid dehydrogenase (3α-HSD) in samples from subjects that have POAG is immunologically distinguishable from the enzyme present in samples from unaffected subjects. In particular, the present inventors have discovered that while certain antibodies react with 3α-HSD in samples from affected and unaffected subjects to similar levels, other antibodies bind to the 3α-HSD in these samples at different levels, thus providing a basis for diagnostic assays. The methods and kits of the invention are described further, as follows.

The diagnostic methods of the invention involve the use of one or more antibodies that are specific for a particular form of 3α-HSD. For example, an antibody that is specific for the form of 3α-HSD that is found in subjects who do not have and are not at risk of developing POAG can be used. In methods employing such an antibody, detection of decreased levels of immunoreactivity in a test sample from a subject, relative to a normal control, provides an indication that the subject has or is at risk of developing POAG. In another example of the methods of the invention, an antibody that is specific for the form of 3α-HSD that is found in subjects who have or are at risk of developing POAG is used. In such methods, detection of increased levels immunoreactivity in a test sample from a subject, relative to a normal control, provides an indication that the subject has or is at risk of developing POAG.

The controls used in the methods described above can be, for example, a similar sample from a subject who is known not to have or be at risk of developing POAG (i.e., a “normal” control). Alternatively, the control can be a sample from a subject known to have or at risk of developing POAG. In addition, any other type of sample that is known to include a particular amount of 3α-HSD that has been determined to be diagnostic relative to a particular type and amount of test sample to be analyzed can be used. Thus, the control can be, e.g., a purified or unpurified preparation of the protein from a different source or a protein produced using recombinant means. Controls can also be 3α-HSD fragments or other peptides that include diagnostic epitopes.

It is possible to omit the use of a control in carrying out the methods of the invention, provided that a level of detection that is diagnostic has been previously determined, and the assay used is designed to be sensitive to this level. For example, in the case of an assay employing an antibody that is tagged with a visually detectable label, the assay can be designed so that the sensitivity is such that any detection of a signal by a person of ordinary visual acuity or a device set at a predetermined level can be relied upon as being diagnostic, provided that the proper type and amount of sample is used. Details of these and other types of assays that can be used in the invention are provided below.

Any of a number of test formats that are known in the art can be used in the invention. For example, as is described in the experimental results section, below, a Western blot format can be used. As is known in the art, this type of assay generally involves the fractionation of a protein-containing sample (e.g., a patient sample, see below) by polyacrylamide gel electrophoresis, transfer of the gel-fractionated proteins onto a membrane, probing of the membrane with an antibody, and detection of any antibody that is specifically bound to the membrane. Quantification of the level of antibody that is specifically bound to the appropriate band on the membrane can be carried out using standard methods, such as densitometry. Details of results obtained using this format with venous blood samples are provided below.

Another example of a type of assay format that can be used in the invention is the enzyme-linked immunosorbent assay (ELISA). In one of many possible examples of this assay format, an antibody that is specific for a particular form of 3α-HSD (see above) is bound to the well of an assay plate, a test sample is added to the well and, after washing, the well is contacted with an enzyme-labeled antibody that binds to both forms of 3α-HSD. After unbound antibody is washed away, the level of antibody bound to the well is detected by, e.g., addition of a calorimetric substrate for the enzyme label. Alternatively, if the antibody that binds to both forms of 3α-HSD is not labeled, a detectably labeled secondary antibody that binds to the constant region of the former antibody can be used. Thus, in the case of the use of a format employing an antibody specific for the form of 3α-HSD found in subjects not affected by POAG bound to the assay plate, detection of a signal would indicate that the subject does not have POAG or a likelihood of developing POAG. Conversely, in the case of a format employing an antibody specific for the form of 3α-HSD found in subjects affected by POAG bound to the assay plate, detection of a signal would indicate a diagnosis of POAG or a likelihood of developing POAG. A related assay format, the radioimmunoassay (RIA), can also be used in the invention. This format is similar to the ELISA, except that it employs a radiolabelled ligand in place of the enzyme label noted in the description of the ELISA.

In another example of an assay format that can be used in the invention, an adherent medium, such as a plastic strip, containing surface bound antibody specific for a particular form of 3α-HSD (see above) is contacted with an appropriate dilution of a patient sample, and any 3α-HSD of the appropriate form that is present in the sample is detected by binding to the medium. In such a format, the concentration of the patient sample is adjusted, if needed, so that the threshold of detection corresponds to a diagnostic level. After incubation with the sample, the strip is washed, and then contacted with a labeled (e.g., enzyme-labeled) antibody that binds to all forms of 3α-HSD, the strip is washed again, a substrate for the enzyme is added, and the level of antibody bound to the medium is detected by measurement of a reaction product of the enzyme and substrate.

Another example of an assay format that can be used in the invention involves the use of a chromatographic medium, such as a paper strip, that contains surface bound antibodies specific for a particular form of 3α-HSD at or near one end. This end of the strip is contacted with a sample from a patient, and it is determined whether a complex forms between the antibody and any 3α-HSD of the form corresponding to the antibody in the sample by allowing chromatography to proceed and assessing the migration of any complexes formed. For example, a detectably labeled antibody can be used which, when bound to ligand, will migrate differently than when unbound. Thus, in this example, it may be that two bands of antibody migration will possibly be detected, which correspond to bound and unbound antibody. In another example, a solution containing the antibody is mixed with a test sample, and the mixture is contacted with the test strip. In any case, the test strips can be contained within a plastic housing that allows contact of the sample or the mixture with the test strip and readout of results. The formatting of such test assays is well known in the art.

The invention also includes kits that can be used to conduct any of the methods described above. Such kits can include, for example, one or more of the 3α-HSD-specific antibodies described above. Further, the kits can optionally include strips of adherent medium, chromatography strips, or polystyrene plates to which antibodies useful in the invention are or can be bound. The kits can also include one or more control protein samples and/or antibodies, such as 3α-HSD standards in differing amounts. Further, the kits can include substrates for any enzyme-labeled antibodies, as well as instructions for carrying out the assays.

Any of a number of appropriate patient samples can be selected for analysis according to the methods of the invention. For example, whole blood, plasma, serum, blood cells (e.g., lymphocytes), urine, or saliva samples can be used. Additional types of patient samples that can be tested include cell or tissue samples, such as cell scrapings (e.g., epithelial cells scraped from the inside lining of the cheek). Standard methods for obtaining and, if necessary, processing such samples for use in diagnostic methods are well known in the art. As a specific example, which is described in further detail below, venous blood samples can be obtained from patients, and peripheral blood lymphocytes isolated by standard ficoll centrifugation. Depending on the source of the sample, the parameters of the assays can be adjusted to the appropriate level of sensitivity, as can readily be carried out by those of skill in the art.

Antibodies that can be used in the methods and kits of the invention include, for example, polyclonal, monospecific, monoclonal, single chain, recombinant, chimeric, and humanized antibodies, as well as antibody fragments (e.g., F(ab′)2, Fab′, Fab, Fv, and sFv fragments) having the specificity and effector functions required for use in the present invention. The antibodies can be of any class determined to be appropriate for use in a given test format. For example, antibodies of IgG, IgA, IgM, IgE, or IgD classes, and subclasses thereof, can be used. Methods for obtaining or synthesizing such antibody or antibody-like molecules are well known in the art (see, e.g., Harlow and Lane, “Using Antibodies—A Laboratory Manual,” Cold Spring Harbor Laboratory Press, Cold Spring Harbor, N.Y., 1998). The antibodies can be tested for appropriate specificity using, for example, the screening methods described herein.

Antigens used to generate antibodies for use in the invention can be purified or partially purified 3α-HSD, as well as 3α-HSD produced using standard recombinant methods. Alternatively, fragments or fusion proteins including 3α-HSD sequences can be used, as determined to be appropriate by those of skill in the art. In the case of a purified protein, the antigen can be obtained from a human sample or, alternatively, from another species.

As is noted above, the invention also includes methods for identifying agents that can be used in the prevention and treatment of POAG, as well as methods for preventing and treating POAG using such agents. The screening methods of the invention involve contacting a cell, such as a trabecular meshwork (TM) cell, with a candidate agent and determining whether the agent affects the immunoreactivity of an antibody specific for 3α-HSD with 3α-HSD of the cell. An agent is identified as being useful for the prevention or treatment of POAG if it decreases immunoreactivity of an antibody specific for the form of 3α-HSD present in subjects who have or are at risk of developing POAG with 3α-HSD in the cell, or if it increases immunoreactivity of an antibody specific for the form of 3α-HSD present in unaffected subjects with 3α-HSD in the cell. In these methods, the cell can be a cultured TM cell, e.g., a cultured human or bovine TM cell, whether primary or immortalized (see, e.g., Polansky et al., Ophthalmology 91(6):580-595, 1984; Kawa et al., Exp. Eye Res 57(5):557-565, 1993; Pang et al., Curr. Eye Res. 13(1):51-63, 1994; Liu et al., Curr. Eye Res. 25(6):347-353, 2002). Alternatively, the cell can be present in any of a number of animal models of POAG that are known in the art. As specific examples, monkeys with glaucoma caused by laser treatment of the trabecular meshwork, or rats with glaucoma induced by destruction of episcleral veins can be used.

In other screening methods of the invention, an animal model of POAG is contacted with a candidate agent, and the impact of the agent on the immunoreactivity of an antibody specific for 3α-HSD on 3α-HSD present in a sample (e.g., a blood sample, such as whole blood or peripheral blood lymphocytes) of the animal is determined. An agent is identified as being useful for the prevention or treatment of POAG if it decreases immunoreactivity of 3α-HSD in the sample to an antibody specific for the form of 3α-HSD present in subjects who have or are at risk of developing POAG, or if it increases immunoreactivity of 3α-HSD in the sample to an antibody specific for the form of 3α-HSD present in unaffected subjects. Candidate compounds that can be tested in the screening methods described above include, for example, small molecules (organic or inorganic), proteins (e.g., peptides or antibodies), amino acids, nucleic acid molecules (e.g., antisense or RNA interference (RNAi) nucleic acid molecules), and carbohydrates.

The invention also includes methods for preventing or treating POAG in patients by use of agents such as those identified using the methods described above. Such agents can be administered using methods determined to be appropriate by those of skill in the art. For example, the agents can be formulated as solutions in isotonic, pH adjusted, sterile saline, optionally in combination with a preservative (e.g., a benzylalkonium chloride), and administered in the form of an eye drop. Alternatively, they can be formulated in an ointment for application to the eye, such as petroleum. Other modes of administration, such as oral and parenteral modes, can also be used. Formulation of agents for administration by these and other appropriate routes are well known in the art (see, e.g., Remington: The Science and Practice of Pharmacy (20^{th} ed., ed. A. R. Gennaro AR.), Lippincott Williams & Wilkins, 2000). Appropriate amounts of the agents to be used can be determined using routine optimization techniques that are dependent on, for example, the condition of the subject, the route of administration, the formulation, the judgment of the practitioner, and other factors evident to those skilled in the art. In general, the agents are administered in amounts ranging from, for example, 1 μg to 500 mg, e.g., about 10 μg to about 100 mg, 100 μg to 50 mg, or 1 mg to 10 mg. The dosage can be administered in a single dose, a divided daily dose, or multiple daily doses, as determined to be appropriate by those of skill in this art.

The invention is based, in part, on the following experimental results.

Venous blood was collected from subjects (POAG and nonglaucomatous controls) and peripheral blood lymphocytes (PBL) were isolated from the blood using the standard “ficoll centrifugation method,” washed, and then frozen at −80° C. Several samples were then analyzed by Western blot analysis, using 3α-HSD antiserum. The antiserum was prepared from rabbits that had been immunized with purified rat liver 3α-HSD. The details of this analysis are as follows.

Venous blood was collected in vacuum tubes containing EDTA as an anticoagulant. About ten ml of blood was diluted with an equal volume of phosphate buffered physiologic saline, without calcium or magnesium (PBS). The diluted blood was then layered over 15 ml of Ficoll-Paque PLUS (Amersham Pharmacia Biotech AB) and subjected to centrifugation in a swinging bucket rotor for 40 minutes at 400× gravity. The cell layer that formed at the ficoll interface was removed with a pipette, diluted with 5 volumes of PBS, and washed two times in PBS by centrifugation at 100× gravity for 10 minutes. The final cell pellet, containing peripheral blood lymphocytes, was frozen at −80° C. until further use.

The frozen cells were defrosted in 3 times their volume of 20% SDS, 50 mM Tris HCl, pH 7.5, 10 mM DTT, heated for 3 minutes at 100° C., and the viscosity of the sample was then reduced by pulse sonification in a cup horn at maximum power to shear DNA. Aliquots of the sample were organic phase precipitated by the addition of 4:1 methanol/chloroform, resuspended in 0.1 ml 0.3% SDS, 50 mM Tris HCl, pH 8.0, 10 mM DTT, incubated at room temperature (RT; about 23° C.) with 0.1 mg/ml DNAse I, 2.5 mg/ml pancreatic RNAse, heated to 100° C. for 3 minutes, alkylated by reaction with 0.05 M iodoacetamide, and reprecipitated by organic phase. The precipitate was resuspended in Laemmlli sample buffer and proteins in the sample were separated by polyacrylamide gel electrophoresis on a 12% gel containing SDS.

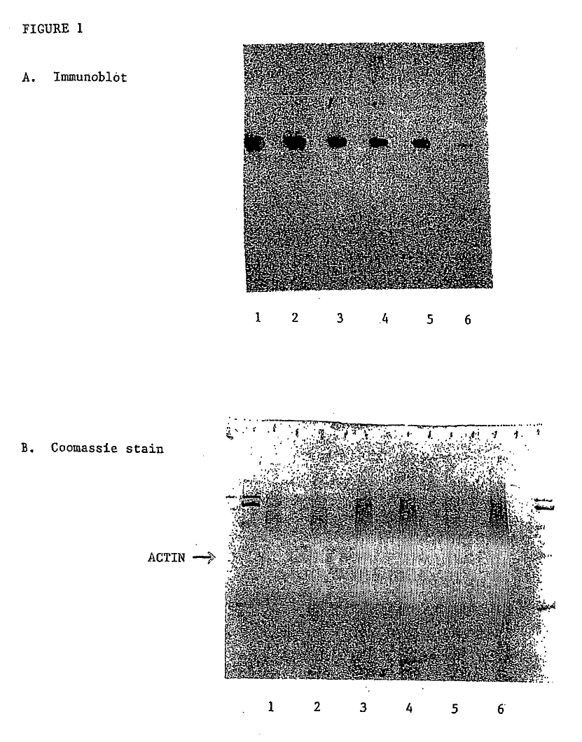

Parallel gels were processed: one was stained for protein with Coomassie blue and the other was transferred to a PVDF membrane in 20 mM CAPS, pH 11, 10% methanol, at 120 mA for 45 minutes. After transfer, the membrane was blocked at 37° C. for 2 hours with 2% casein, 50 mM Tris HCl, pH 7.5, 150 mM NaCl, 0.1% Na Azide, and incubated with 3α-HSD polyclonal rabbit antiserum diluted 1:5,000 in the casein buffer for 18 hours at RT. After washing in 0.05% Tween 20 containing 50 mM Tris HCl, pH 7.5, 150 mM NaCl (TTBS), a second antibody (horseradish peroxidase-linked goat anti-rabbit antibody (1:30,000)) was incubated with the blot in 1% gelatin TTBS for 1 hour at RT. After more washing in TTBS, the bound second antibody was visualized following hydrogen peroxide and dye treatment with standard photographic techniques using the Pierce detection system. The intensities of the signal were quantified by densitometry and normalized to the amount of actin present in the samples, as determined by scanning the Coomassie stained bands on the parallel gel. FIG. 1A shows the results of immunostaining of the blot, with lanes 1-3 containing the non-POAG samples and lanes 4-6 containing the POAG samples. FIG. 1B is the parallel gel that was stained with Coomassie blue. The digitized values of the bands were determined using the software program, Immagequant (Amersham), and the amount of immunostaining of 3α-HSD was normalized to the amounts of actin detected on the stained gel. The POAG derived cells contained on average a 5-fold reduction of 3α-HSD antigen (in relative units: 0.20, 0.17, and 0.01 for POAG cells as compared to 1.0, 0.79, and 0.45 for control cells).

Our previous studies demonstrated that 3α-HSD activity is reduced in POAG derived cell preparations. We determined by DNA sequencing that this reduced activity was not due to a mutation(s) in the coding sequence for this enzyme. In addition, quantitative RT PCR analysis demonstrated no significant differences in 3α-HSD mRNA levels between POAG and non-POAG derived cells. In other studies, using a different polyclonal antibody to 3α-HSD than that used in the experiments described above, we found that there was no change in immunoreactivity between POAG and non-POAG derived cells. Based on these data, we conclude that the currently observed changes in immunoreactivity in POAG samples is not due to a decrease in protein amount but rather, is due to a post-translational modification of the protein that masks an epitope in the POAG-derived protein. It is this modification that provides a basis for immunological distinction between 3α-HSD from patients vs. unaffected subjects, which in turn provides the basis for the diagnostic methods described herein.

What is claimed is:

1. A method of determining whether a subject has or is at risk of developing primary open angle glaucoma (POAG), the method comprising contacting a sample from the subject with an antibody specific for 3α-hydroxysteroid dehydrogenase (3α-HSD), and classifying the subject as having or being at risk for developing POAG or not, based on the level of immunoreactivity of the antibody with 3α-HSD present in the sample.

2. The method of claim 1, wherein the antibody is specific for the form of 3α-HSD present in subjects who have or are at risk of developing POAG, and detection of increased immunoreactivity in the sample with the antibody, relative to a control based on the form of 3α-HSD present in unaffected subjects, indicates a diagnosis of POAG or a risk thereof;
the antibody is specific for the form of 3α-HSD present in subjects who have or are at risk of developing POAG, and detection of decreased immunoreactivity in the sample with the antibody, relative to a control based on the form of 3α-HSD present in subjects who have or are at risk of developing POAG, indicates that the subject does not have and is not at risk of developing POAG;
the antibody is specific for the form of 3α-HSD present in subjects who do not have or are not at risk of developing POAG, and detection of decreased immunoreactivity in the sample with the antibody, relative to a control based on the form of 3α-HSD present in unaffected subjects, indicates a diagnosis of POAG or a risk thereof, or
the antibody is specific for the form of 3α-HSD present in subjects who do not have or are not at risk of developing POAG, and detection of increased immunoreactivity in the sample with the antibody, relative to a control sample based on the form of 3α-HSD present in subjects who have or are at risk of developing POAG, indicates that the subject does not have and is not at risk of developing POAG.

3-5. (canceled)

6. The method of claim 1, wherein a control is processed in parallel with the sample from the subject.

7. The method of claim 1, wherein the sample is fractionated by gel electrophoresis and transferred to a membrane prior to contact with the antibody.

8. The method of claim 1, wherein the antibody is bound to an adherent medium, the sample is contacted with the antibody on the adherent medium, and binding of 3α-HSD of the sample to the antibody on the adherent medium is detected by use of a detectably labeled antibody that specifically binds to 3α-HSD.

9. The method of claim 8, wherein the adherent medium is a well of an assay plate or a plastic strip.

10. (canceled)

11. The method of claim 1, wherein any complexes formed between the antibody and 3α-HSD in the sample are detected after chromatographic separation of the complexes from uncomplexed antibody.

12. The method of claim 1, wherein the sample is selected from the group consisting of whole blood, plasma, serum, blood cells, peripheral blood lymphocytes, urine, saliva, and epithelial cells.

13. (canceled)

14. A kit for determining whether a subject has or is at risk of developing POAG, the kit comprising an antibody specific for 3α-HSD.

15. The kit of claim 14, wherein the antibody is specific for the form of 3α-HSD present in subjects who have or are at risk of developing POAG, or is specific for the form of 3α-HSD present in subjects who do not have or are not at risk of developing POAG.

16-17. (canceled)

18. The kit of claim 14, further comprising a standard comprising 3α-HSD in the form that is present in subjects who do not have or are not at risk of developing POAG or a standard comprising 3α-HSD in the form that is present in subjects who have or are at risk of developing POAG.

19-20. (canceled)

21. The kit of claim 14, further comprising an adherent medium to which the antibody binds.

22. The kit of claim 21, wherein the adherent medium is an assay plate or a plastic strip.

23. (canceled)

24. The kit of claim 14, further comprising a chromatographic medium for distinguishing complexes formed between 3α-HSD and the antibody and free antibody.

25. The kit of claim 14, further comprising a detectably labeled secondary antibody.

26. A method of identifying an agent that can be used in the prevention or treatment of POAG, comprising contacting a trabecular meshwork cell from a subject with POAG with a candidate agent and classifying the agent as being effective in the treatment or prevention of POAG, based on the effect of the agent on the level of immunoreactivity of an antibody specific for 3α-HSD with 3α-HSD of the cell.

27. The method of claim 26, wherein the antibody specific for 3α-HSD is specific for the form of 3α-HSD present in subjects who have or are at risk of developing POAG, and detection of a decreased level of immunoreactivity of the antibody with 3α-HSD of the cell, in the presence of the candidate agent, indicates the identification of an agent that can be used in the prevention or treatment of POAG; or
the antibody specific for 3α-HSD is specific for the form of 3α-HSD present in subjects who do not have or are not at risk of developing POAG, and detection of an increased level of immunoreactivity of the antibody with 3α-HSD of the cell, in the presence of the candidate agent, indicates the identification of an agent that can be used in the prevention or treatment of POAG.

28. (canceled)

29. The method of claim 26, wherein the trabecular meshwork cell is a cultured cell or is present in an animal.

30. (canceled)

31. A method of identifying an agent that can be used in the prevention or treatment of POAG, comprising administering a candidate agent to a subject having POAG or a model condition thereof, and classifying the agent as being effective in the treatment or prevention of POAG, based on the effect of the agent on the level of immunoreactivity of an antibody specific for 3α-HSD with a sample from the subject.

32. The method of claim 31, wherein the antibody specific for 3α-HSD is specific for the form of 3α-HSD present in subjects who have or are at risk of developing POAG, and detection of a decreased level of immunoreactivity of the antibody with 3α-HSD in the sample, in the presence of the candidate agent, indicates the identification of an agent that can be used in the prevention or treatment of POAG or
the antibody specific for 3α-HSD is specific for the form of 3α-HSD present in subjects who do not have or are not at risk of developing POAG, and detection of an increased level of immunoreactivity of the antibody with 3α-HSD in the sample, in the presence of the candidate agent, indicates the identification of an agent that can be used in the prevention or treatment of POAG.

33. (canceled)

34. The method of claim 31, wherein the subject is an animal model of human POAG.

35. The method of claim 31, wherein the sample is selected from the group consisting of whole blood, plasma, serum, blood cells, peripheral blood lymphocytes, urine, saliva, and epithelial cells.

36. (canceled)

37. A method of preventing or treating POAG in a subject, the method comprising administering to the subject an agent identifiable as being effective using the method of claim 26.

38. The method of claim 37, wherein the agent is administered to the ocular surface of the subject.

39. A method of preventing or treating POAG in a subject, the method comprising administering to the subject an agent identifiable as being effective using the method of claim 31.

40. The method of claim 39, wherein the agent is administered to the ocular surface of the subject.
